(19) United States
(12) Patent Application Publication
(10) Pub. No.: US 2025/0253059 A1
(43) Pub. Date: Aug. 7, 2025

(54) DIGITAL PHENOTYPING METHOD, APPARATUS, AND COMPUTER PROGRAM FOR DRUG RESPONSE CLASSIFICATION AND PREDICTION

(71) Applicant: IMEDISYNC, INC., Seoul (KR)

(72) Inventor: Seungwan KANG, Seoul (KR)

(73) Assignee: IMEDISYNC, INC., Seoul (KR)

(21) Appl. No.: 19/185,734

(22) Filed: Apr. 22, 2025

Related U.S. Application Data
(63) Continuation of application No. PCT/KR2022/017089, filed on Nov. 3, 2022.

(30) Foreign Application Priority Data
Oct. 25, 2022 (KR) 10-2022-0138000

(51) Int. Cl. G16H 50/50; A61B 5/00; A61B 5/372; G16H 50/20

(52) U.S. Cl. CPC G16H 50/50; A61B 5/372; A61B 5/4088; A61B 5/4848; A61B 5/7246; A61B 5/7267; G16H 50/20

(57) ABSTRACT

Provided are a digital phenotyping method, apparatus, and recording medium for drug response classification and prediction. A digital phenotyping method for drug response classification and prediction that is performed by a computing device according to various embodiments of the present invention includes acquiring biometric data of a patient, and analyzing the acquired biometric data using a disease diagnostic model to perform a multiple disease diagnosis on the patient, in which the disease diagnostic model includes a plurality of diagnostic models that independently perform diagnoses of each of multiple distinct diseases based on the acquired biometric data.

### CROSS-REFERENCE TO RELATED APPLICATIONS

This application is a Continuation of International Application No. PCT/KR2022/017089, filed on Nov. 3, 2022, which claims priority to and the benefit of Korean Patent Application No. 10-2022-0138000, filed on Oct. 25, 2022, the disclosure of which is incorporated herein by reference in its entirety.

### BACKGROUND

### 1. Field of the Invention

Various embodiments of the present invention relate to a digital phenotyping method, apparatus, and recording medium for drug response classification and prediction.

### 2. Discussion of Related Art

Dementia refers to a series of symptoms caused by brain disease.

Dementia is a state in which the ability to perform daily activities is impaired due to a decline in cognitive ability. As the dementia progresses, it affects thinking ability, behavior, and performance of daily life. Doctors diagnose dementia when two or more cognitive functions (e.g., memory, language function, information comprehension, spatial function, judgment, and attention) are significantly impaired.

Dementia patients may have difficulty for solving problems and controlling emotions and experience personality changes. Exact symptoms experienced by dementia patients depend on which part of a brain is damaged by diseases causing dementia. In various types of dementia, some of brain's nerve cells stop functioning and die because of the loss of connections to other cells. The dementia generally progresses steadily. That is, the dementia gradually spreads throughout the brain, and symptoms of patients worsen over time.

In Korea, about 290,000 people, which is approximately 9.5% of the elderly population aged 65 or older, are suffering from senile dementia, and 180,000 people, which is 73% of them, are severe patients who habitually wander streets. The number of patients with senile dementia is expected to continuously increase as the aging of the population accelerates in the future.

Alzheimer disease dementia (ADD) is the most common form of dementia, and about 70% or more of dementia patients suffer from ADD. Meanwhile, as a result of pathologically dissecting brains of patients with ADD after death, patients with cells called Lewy body were found, and about 20% of the patients are called Lewy body dementia (LBD).

A patient with ADD mixed with LBD (ADD dominant LBD mix) typically experience faster disease progression and a greater decline in cognitive function compared to patients with ADD not mixed with LBD.

In addition, Aducanumab, which has a mechanism to remove Amyloid-beta, a protein known as the cause of dementia, Lecanemab, which has reduced the side effects of Aducanumab, etc., are representative dementia treatments used to treat ADD. The patient with the ADD mixed with the LBD (ADD dominant LBD mix) has the characteristic that the cognitive function does not improve even after removing the Amyloid-beta.

In other words, even if a patient is a dementia patient, dementia treatments and treatment methods differ depending on whether a dementia patient is a patient with pure-ADD or the ADD dominant LBD mix. Therefore, in order to provide accurate treatment and medication for dementia patients, it is necessary to accurately determine whether the dementia patient simply has the ADD or has the ADD dominant LBD mix. However, the problem is that the conventional dementia patient diagnosis method does not accurately classify dementia patients.

In addition, in the process of developing pharmaceuticals such as dementia treatment drugs, it is necessary to accurately classify a type of dementia of dementia patients in order to select only a pure-ADD group not mixed with LBD as clinical subjects and to prescribe the developed dementia treatment for pure-ADD not mixed with LBD.

### SUMMARY OF THE INVENTION

The problem to be solved by the present invention intends to solve the problems of the conventional dementia patient diagnosis method described above, and the present invention is directed to providing a digital phenotyping method, apparatus, and recording medium for drug response classification and prediction which are capable of more accurately classifying a type of dementia of a dementia patient by analyzing biometric data of a patient to determine whether the patient has Alzheimer disease dementia (ADD), and also determine whether the patient has Lewy body dementia (LBD) as well as whether the patient has ADD.

The present invention is directed to providing a digital phenotyping method, apparatus, and recording medium for drug response classification and prediction which are capable of analyzing a biometric data of a patient through a plurality of different diagnostic models to independently and simultaneously determine not only whether a patient has dementia but also to perform different types of brain diseases such as dementia with Lewy body, Parkinson, vascular dementia, depression, and anxiety.

Problems of the present invention are not limited to the above-described problems. That is, other problems that are not described may be obviously understood by those skilled in the art from the following description.

According to an aspect of the present invention, there is provided a digital phenotyping method for drug response classification and prediction that is performed by a computing device, which includes acquiring biometric data of a patient, and analyzing the acquired biometric data using a disease diagnostic model to perform a multiple disease diagnosis on the patient, in which the disease diagnostic model includes a plurality of diagnostic models that independently perform diagnoses of each of multiple distinct diseases based on the acquired biometric data.

The digital phenotyping method may further include acquiring a plurality of pieces of brainwave data for each of multiple patients as biometric data of the multiple patients having different types of brain diseases, classifying the plurality of pieces of acquired brainwave data based on a type of brain disease, and generating a plurality of diagnostic models that individually diagnose whether to have different types of brain diseases by training different diagnostic models using the plurality of pieces of classified brainwave data as training data.

The acquiring of the plurality of pieces of brainwave data may include acquiring first brainwave data for a patient with a specific brain disease at a first time point, which is a time point before the patient with the specific brain disease takes a target drug, and acquiring second brainwave data for the patient with the specific brain disease at a second time point, which is a time point after the patient with the specific brain disease takes the target drug, and the generating of the plurality of diagnostic models may include comparing the acquired first brainwave data with the acquired second brainwave data to calculate a validity value, classifying the acquired first brainwave data into valid first brainwave data when the calculated validity value is greater than or equal to a preset validity value, and training a diagnostic model, which diagnoses whether the patient has the specific brain disease, among the plurality of generated diagnostic models using the classified valid first brainwave data as the training data.

The plurality of diagnostic models may include a first diagnostic model for diagnosing whether a first disease is present and a second diagnostic model for diagnosing whether a second disease related to the first disease is present, and the performing of the multiple disease diagnosis may include calculating a first probability value, which is a possibility that the patient has the first disease, by analyzing the acquired biometric data through the first diagnostic model, when a request for diagnosis of the first disease for the patient is acquired from a user, and when the calculated first probability value is greater than or equal to a reference probability value, calculating a second probability value, which is a possibility that the patient has the second disease, by analyzing the acquired biometric data through the second diagnostic model, and performing multiple diagnoses of whether the first disease is present and whether the second disease is present based on the calculated first probability value and the calculated second probability value.

The performing of the multiple diagnoses may include determining that the patient has only the first disease when the calculated second probability value is less than the reference probability value and determining that the patient has the first disease and the second disease when the calculated second probability value is greater than or equal to the reference probability value.

The determining of that the patient has the first disease and the second disease may include determining a dominant between the first disease and the second disease based on a result of comparing the calculated first probability value and the calculated second probability value and a difference between the calculated first probability value and the calculated second probability value, determining that the patient has the first disease mixed with a symptom of the second disease based on the determined dominant, when the calculated first probability value is greater than the calculated second probability value and the difference between the calculated first probability value and the calculated second probability value is greater than or equal to a preset difference value, determining that the patient has both the first disease and the second disease based on the determined dominant when a magnitude of the difference between the calculated first probability value and the calculated second probability value is less than the preset difference value, and determining that the patient has the second disease mixed with a symptom of the first disease based on the determined dominant, when the calculated second probability value is greater than the calculated first probability value and a difference between the calculated second probability value and the calculated first probability value is greater than or equal to the preset difference value.

The performing of the multiple disease diagnosis may include calculating a probability value corresponding to the possibility that the patient has each of the multiple distinct diseases by inputting the acquired biometric data to each of the plurality of diagnostic models, and selecting at least one disease of which a calculated probability value is greater than or equal to a reference probability value from among the multiple distinct diseases, and determining that the patient is a patient having at least one of the selected diseases as a result of the multiple disease diagnosis of the patient.

The performing of the multiple disease diagnosis may include selecting at least one second disease having a correlation with the first disease based on a plurality of predefined correlations between diseases when acquiring a first disease diagnostic request for the patient from a user, and calculating a first probability value that is a possibility of having the first disease and one or more second probability values that is a possibility of having the selected one or more second diseases by analyzing the acquired biometric data through one diagnostic model that performs a diagnosis of the first disease among the plurality of diagnostic models and one or more diagnostic models that perform a diagnosis of the selected one or more second diseases.

The performing of the multiple disease diagnosis may include calculating a plurality of probability values, which are possibilities of having each of the multiple distinct diseases, by analyzing the acquired biometric data through the plurality of diagnostic models, grouping the plurality of calculated probability values according to correlations based on a plurality of predefined correlations between diseases, and performing the multiple disease diagnosis on the patient based on a comparison result between each of the plurality of grouped probability values and a reference probability value, a size comparison result between the plurality of grouped probability values, and a difference between the plurality of grouped probability values.

According to another aspect of the present invention, there is provided a digital phenotyping method for drug response classification and prediction that is performed by a computing device, which includes acquiring first biometric data of a patient at a first time point which is a time point before the patient takes a target drug, acquiring second biometric data of the patient at a second time point which is later than the first time point and is a time point after the patient takes the target drug, and analyzing the acquired first biometric data using a disease diagnostic model to perform a multiple disease diagnosis on the patient, in which the disease diagnostic model may include a plurality of diagnostic models that independently perform diagnoses of each of multiple distinct diseases based on the acquired biometric data.

The digital phenotyping method may further include acquiring first brainwave data measured at the first time point and second brainwave data measured at the second time point for each of the multiple patients as biometric data of multiple patients having different types of brain diseases, classifying the acquired first brainwave data based on a type of brain disease, and generating a plurality of diagnostic models that individually diagnose whether different types of brain diseases are present by training different diagnostic models using the classified first brainwave data as training data.

The digital phenotyping method may further include calculating a validity value through a comparison between the acquired first brainwave data and second brainwave data, classifying the acquired first brainwave data into valid first brainwave data when the calculated validity value is greater than or equal to a preset validity value, and regenerating the plurality of diagnostic models using the valid first brainwave data as the training data.

According to still another aspect of the present invention, there is provided a digital phenotyping apparatus for drug response classification and prediction, which includes a processor, a network interface, a memory, and a computer program loaded into the memory and executed by the processor, in which the computer program includes an instruction for acquiring biometric data of a patient, and an instruction for analyzing the acquired biometric data using a disease diagnostic model to perform a multiple disease diagnosis on the patient, and the disease diagnostic model includes a plurality of diagnostic models that independently perform diagnoses of each of multiple distinct diseases based on the acquired biometric data.

According to still yet another aspect of the present invention, there is provided a computer program stored in a computer-readable recording medium, which is combined with a computing device to execute a digital phenotyping method for drug response classification and prediction, in which the digital phenotyping method includes acquiring biometric data of a patient, and analyze the acquired biometric data using a disease diagnostic model to perform a multiple disease diagnosis on the patient, the disease diagnostic model including a plurality of diagnostic models that independently perform diagnoses of each of multiple distinct diseases based on the acquired biometric data.

Other detailed contents of the present invention are described in a detailed description and are illustrated in the drawings.

### BRIEF DESCRIPTION OF DRAWINGS

The above and other objects, features and advantages of the present invention will become more apparent to those of ordinary skill in the art by describing exemplary embodiments thereof in detail with reference to the accompanying drawings, in which:

FIGS. 1 to 3 are diagrams illustrating results of a comparative experiment between Alzheimer disease dementia (pure-ADD) patients and Lewy body dementia (pure-LBD) patients;

FIG. 4 is a diagram illustrating a digital phenotyping system for drug response classification and prediction according to an embodiment of the present invention;

FIG. 5 is a hardware configuration of a digital phenotyping apparatus for drug response classification and prediction according to another embodiment of the present invention;

FIG. 6 is a flowchart of a digital phenotyping method for drug response classification and prediction according to a first embodiment of the present invention;

FIG. 7 is a diagram illustrating a process of performing a multiple disease diagnosis using a disease diagnostic model including a plurality of diagnostic models in the first embodiment;

FIG. 8 is a flowchart for describing a method of generating a plurality of diagnostic models in the first embodiment;

FIG. 9 is a flowchart of a digital phenotyping method for drug response classification and prediction according to a second embodiment of the present invention;

FIG. 10 is a flowchart for describing a method of generating a plurality of diagnostic models in the second embodiment;

FIG. 11 is a flowchart for describing a method of regenerating a plurality of diagnostic models in the second embodiment;

FIG. 12 is a flowchart for describing a method of sequentially performing a diagnosis of a first disease and a second disease having a correlation in various embodiments;

FIG. 13 is a flowchart for describing a method of simultaneously performing a diagnosis of a first disease and a second disease having a mutual correlation in various embodiments; and

FIG. 14 is a flowchart for describing a method of simultaneously performing a diagnosis of multiple diseases in various embodiments.

### DETAILED DESCRIPTION OF EXEMPLARY EMBODIMENTS

Advantages and features of the present disclosure and methods to achieve them will be elucidated from embodiments described below in detail with reference to the accompanying drawings. However, the present disclosure is not limited to embodiments to be described below, but may be implemented in various different forms, these embodiments will be provided only in order to make the present disclosure complete and allow those skilled in the art to completely recognize the scope of the present disclosure, and the present disclosure will be defined by the scope of the claims.

Terms used herein are for describing embodiments rather than limiting the present disclosure. Unless explicitly described to the contrary, a singular form includes a plural form in the present specification. The terms “comprise” and/or “comprising” used in the present disclosure do not exclude the existence or addition of one or more other components other than the mentioned components. Throughout the present disclosure, the same components will be denoted by the same reference numerals, and the term “and/or” includes each and all combinations of one or more of the mentioned components. The terms “first,” “second,” and the like are used to describe various components, but these components are not limited by these terms. These terms are used only in order to distinguish one component from other components. Accordingly, a first component mentioned below may be a second component within the technical spirit of the present disclosure.

Unless defined otherwise, all terms (including technical and scientific terms) used in the present disclosure have the same meaning as meanings commonly understood by those skilled in the art to which the present disclosure pertains. In addition, terms defined in generally used dictionaries are not ideally or excessively interpreted unless they are specifically defined clearly.

Further, the term “unit” or “module” used herein means a hardware component such as software, FPGA, or ASIC and performs predetermined functions. However, the term “unit” or “module” is not meant to be limited to software or hardware. A “unit” or “module” may be stored in a storage medium that can be addressed or may be configured to regenerate one or more processors. Accordingly, for example, the “unit” or “module” includes components such as software components, object-oriented software components, class components, and task components, processors, functions, attributes, procedures, subroutines, segments of program code, drivers, firmware, a microcode, a circuit, data, a database, data structures, tables, arrays, and variables. Functions provided in components, “units,” or “modules” may be combined into fewer components, “units,” or “modules” or further separated into additional components, “units,” or “modules.”

Spatially relative terms “below,” “beneath,” “lower,” “above,” “upper,” and the like may be used in order to easily describe correlations between one component and other components. The spatially relative terms should be understood as terms including different directions of components during use or operation in addition to the directions illustrated in the drawings. For example, in a case of overturning component illustrated in the drawings, a component described as “below” or “beneath” another component may be placed “above” the other component. Accordingly, the illustrative term “below” may include both of a downward direction and an upward direction. Components may be oriented in other directions as well, and thus, spatially relative terms may be interpreted according to orientations.

In this specification, a computer means all kinds of hardware devices including at least one processor and can be understood as including a software component which is operated in the corresponding hardware device according to the embodiment. For example, the computer may be understood as a meaning including any of smart phones, tablet PCs, desktops, notebooks, and user clients and applications running on each device, but is not limited thereto.

Hereinafter, embodiments of the present invention will be described in detail with reference to the accompanying drawings.

Each step described in this specification is described as being performed by the computer, but subjects of each step are not limited thereto, and according to embodiments, at least part of each step can also be performed on different devices.

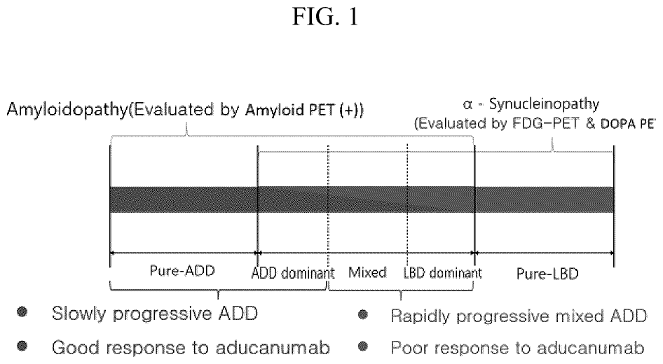

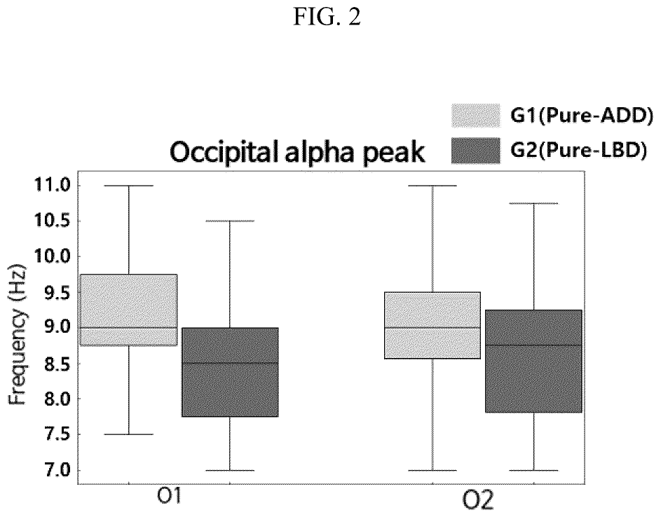

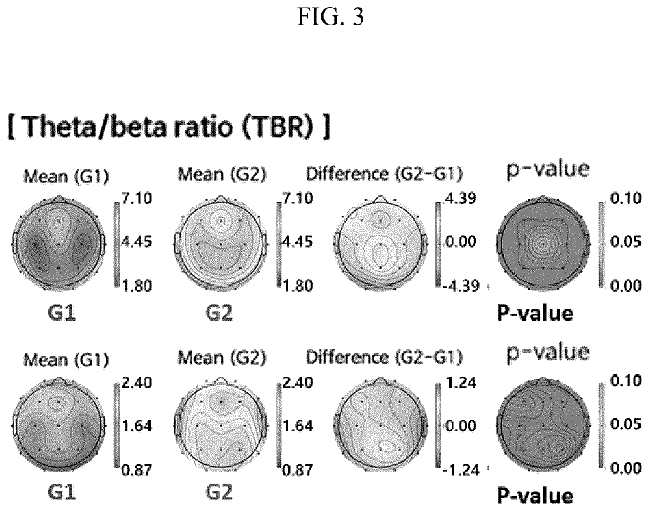

FIGS. 1 to 3 are diagrams illustrating results of a comparative experiment between Alzheimer disease dementia (pure-ADD) patients and Lewy body dementia (pure-LBD) patients.

Referring to FIGS. 1 to 3, pure-LBD, Lewy body dementia patients with which symptoms of Alzheimer disease dementia are mixed (LBD dominant ADD mix), and patients with Alzheimer disease dementia and Lewy body dementia mixed (ADD LBD mixed) have characteristics of faster disease progression and greater decline in cognitive function compared to pure-ADD and patients with which symptoms of Lewy body dementia are mixed (ADD dominant LBD mix).

In addition, the results of comparing brainwave data (brainwave data) of the pure-ADD and the pure-LBD showed that an alpha peak frequency of a pure-LBD group was lower than that of the pure-ADD, and power of a delta frequency and a theta frequency of the pure-LBD group tends to be stronger than that of the pure-ADD.

Here, the alpha peak has individual differences for each person. However, in the case of normal people, the alpha peak is usually formed above 10 Hz and tends to slow down as cognitive impairment occurs. In addition, delta and theta are areas where power increases during sleep, but in the case of normal people without cognitive impairment, the delta and theta waves do not occur significantly. Therefore, it can be seen that the cognitive impairment of the pure-LBD group progressed more than that of the pure-ADD.

Meanwhile, representative dementia treatments include Aducanumab, which has a mechanism to remove Amyloid-beta, a protein known as the cause of dementia, and Lecanemab, which has reduced side effects of Aducanumab. However, the effect of cognitive function improvement due to removal of Amyloid-beta protein may differ depending on the type of dementia (for example, depending on whether a patient has only ADD or has ADD mixed with symptoms of LBD). Therefore, it is necessary to diagnose not only whether the patient has dementia, but also what type of dementia the patient has.

In consideration of this, the digital phenotyping method, apparatus, and recording medium for drug response classification and prediction according to various embodiments of the present invention may perform concomitant diagnosis of multiple diseases on dementia patients so that it may more accurately and specifically determine not only whether a patient has dementia but also what type of dementia a patient has and whether a patient has other diseases, etc.

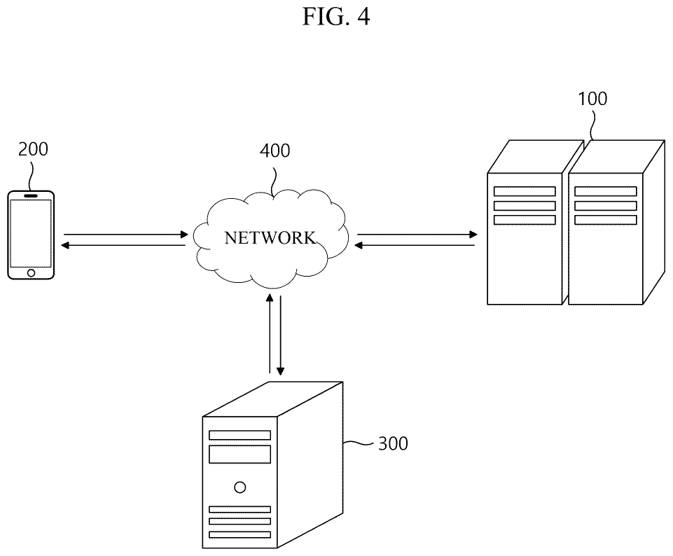

In addition, through such an accurate and specific diagnosis, it is possible to determine whether a dementia patient has other diseases besides ADD, select only the pure-ADD group as a clinical subject during a pharmaceutical development process, and prescribe drugs only for the pure-ADD group. Hereinafter, the digital phenotyping method, apparatus, and recording medium for drug response classification and prediction according to various embodiments of the present invention will be described with reference to FIGS. 4 to 14.

FIG. 4 is a diagram illustrating a digital phenotyping system for drug response classification and prediction according to an embodiment of the present invention.

Referring to FIG. 4, the digital phenotyping system for drug response classification and prediction may include a multiple disease diagnosis device 100, a user terminal 200, an external server 300, and a network 400.

Here, the digital phenotyping system for drug response classification and prediction illustrated in FIG. 4 is according to an embodiment, and components thereof are not limited to the embodiment illustrated in FIG. 4, and some components may be added, changed, or omitted if necessary.

In an embodiment, the multiple disease diagnosis device 100 (hereinafter, “computing device 100”) may analyze biometric data of a patient to perform a multiple disease diagnosis of multiple distinct diseases.

Here, the multiple distinct diseases are different types of brain diseases such as ADD, LBD, Parkinson, vascular dementia, depression, and anxiety, and the biometric data of the patient may be brainwave data necessary for diagnosing the patient's brain disease, but is not limited thereto.

In various embodiments, the computing device 100 may analyze the biometric data of the patient using a disease diagnostic model to perform a multiple disease diagnosis on the patient.

Here, the disease diagnostic model may be a model trained using biometric data labeled with information about diseases (e.g., disease names) of each of multiple patients as training data and may be a model that uses biometric data of a specific patient as input data and outputs information about diseases of a patient or probability values for a specific disease as result data.

The disease diagnostic model (e.g., a neural network) is composed of one or more network functions, and one or more network functions may be composed of a set of interconnected computational units, which may generally be referred to as “nodes.” These “nodes” may also be referred to as “neurons.” One or more network functions include at least one or more nodes. Nodes (or neurons) that constitute one or more network functions may be interconnected by one or more “links.”

Within the disease diagnostic model, one or more nodes connected through the link may form a relative relationship between the input node and output node. The concepts of the input node and the output node are relative, and any node in the relationship of the output node with respect to one node may be in the input node relationship in the relationship with another node, and vice versa. As described above, the relationship between the input node and the output node may be generated around the link. One or more output nodes may be connected to one input node through the link, and vice versa.

In the relationship between the input node and the output node connected through one link, a value of the output node may be determined based on data input to the input node. Here, the node connecting between the input node and the output node may have weights. The weights may be variable and may be varied by a user or algorithm in order for the disease diagnostic model to perform the desired functions. For example, when one or more input nodes are connected to one output node by the respective links, the value of the output node may be determined based on the values input to the input nodes connected to the output node and the weights set on the links corresponding to the respective input nodes.

As described above, the disease diagnostic model interconnects one or more nodes through one or more links to form the relationship between the input node and the output node within the disease diagnostic model. The characteristics of the disease diagnostic model may be determined according to the number of nodes and links within the disease diagnostic model, the correlation between nodes and links, and the weight values assigned to each link. For example, when there are two disease diagnostic models with the same number of nodes and links and different weight values between the links, the two disease diagnostic models may be recognized as different from each other.

Some of the nodes constituting the disease diagnostic model may constitute one layer based on distances from an initial input node. For example, a set of nodes with a distance n from the initial input node may constitute n layers. The distance from the initial input node may be defined by the minimum number of links that should be passed to reach the corresponding node from the initial input node. However, this definition of the layer is arbitrary for explanation purposes, and the order of the layer within the disease diagnostic model may be defined in a different way than described above. For example, the layer of the nodes may be defined by a distance from a final output node.

The initial input nodes may refer to one or more nodes, to which data is directly input without going through links in relationships with other nodes, among the nodes within the disease diagnostic model. Alternatively, the initial input nodes may refer to nodes that do not have other input nodes connected by the link in the relationship between the nodes based on the link within the disease diagnostic model network. Similarly, the final output nodes may refer to one or more nodes that do not have the output node in the relationship with other nodes among the nodes in the disease diagnostic model. In addition, hidden nodes may refer to nodes that constitute the disease diagnostic model rather than the first input node and the last output node. The disease diagnostic model according to an embodiment of the present invention may have more nodes of the input layer than the nodes of the hidden layer close to the output layer and may be the disease diagnostic model in which the number of nodes decreases as it progresses from the input layer to the hidden layer.

The disease diagnostic model may include one or more hidden layers. The hidden node of the hidden layer may use an output of a previous layer and an output of surrounding hidden nodes as an input. The number of hidden nodes for each hidden layer may be the same or different. The number of nodes of the input layer may be determined based on the number of data fields of the input data and may be the same as or different from the number of hidden nodes. The input data input to the input layer may be calculated by the hidden node of the hidden layer and output by a fully connected layer (FCL) which is the output layer.

In various embodiments, the disease diagnostic model may be a deep learning model.

The deep learning model (e.g., a deep neural network (DNN)) may refer to a disease diagnostic model including a plurality of hidden layers in addition to an input layer and an output layer. It is possible to identify latent structures of data by using the DNN. That is, it is possible to identify the latent structures (e.g., what objects are in the photo, what the content and emotion of the text are, what the content and emotion of the audio are, etc.) of a photo, text, video, sound, or music.

The DNN may include a convolutional neural network (CNN), a recurrent neural network (RNN), an auto encoder, a generative adversarial network (GAN), a restricted Boltzmann machine (RBM), a deep belief network (DBN), a Q network, a U network, a Siamese network, etc., but is not limited thereto.

In various embodiments, the network function may include the auto encoder. Here, the autoencoder may be a type of artificial neural network to output the output data similar to the input data.

The autoencoder may include at least one hidden layer, and an odd number of hidden layers may be disposed between the input and output layers. The number of nodes in each layer may be reduced from the number of nodes in the input layer to an intermediate layer called a bottleneck layer (encoding), and then also be scaled up and symmetrically scaled down from the bottleneck layer to the output layer (symmetrical to the input layer). Nodes of a dimension reduction layer and a dimension restoration layer may or may not be symmetrical. In addition, the autoencoder may perform nonlinear dimension reduction. The number of input layers and output layers may correspond to the number of sensors remaining after preprocessing the input data. In the auto encoder structure, the number of nodes in the hidden layer included in the encoder may have a structure that decreases as the distance from the input layer increases. When the number of nodes in the bottleneck layer (the layer with the fewest nodes located between the encoder and the decoder) is too small, a sufficient amount of information may not be transmitted, and therefore, the number of nodes may be maintained at a certain number or more (e.g., more than half of the input layers or the like).

The neural network may be trained by at least one method of supervised learning, unsupervised learning, and semi supervised learning. The training of the neural network is to minimize errors in output. More specifically, the training of the neural network is a process of repeatedly inputting training data to the neural network, calculating an output of the neural network for the training data and target errors, and updating weights of each node of the neural network by back-propagating errors of the neural network from an output layer of the neural network to an input layer in order to reduce the errors.

First, in the case of the supervised learning, training data with a correct answer labeled for each training data is used (i.e., labeled training data), and in the case of the unsupervised learning, a correct answer may not be labeled for each training data. That is, for example, in the case of the supervised learning for data classification, the training data may be data in which each category is labeled for training data. The labeled training data is input to the neural network, and an error may be calculated by comparing the output (category) of the neural network with the label of the training data.

Next, in the case of the unsupervised learning for the data classification, the error may be calculated by comparing the input training data with the output of the neural network. The calculated error is backpropagated in a backward direction (i.e., from an output layer to an input layer) in the neural network, and connection weights of each node in each layer of the neural network may be updated according to the backpropagation. The amount of change in the connection weights of each node to be updated may be determined according to a learning rate. The calculation of the neural network for the input data and the backpropagation of the error may constitute a learning cycle (epoch). The learning rate may be applied differently depending on the number of times of repetitions of the learning cycle of the neural network. For example, in the early stage of the training of the neural network, a high learning rate may be used to allow the neural network to quickly acquire a certain level of performance, thereby increasing efficiency, and in the later stage of the training, a low learning rate may be used to increase accuracy.

In the training of the neural network, the training data may generally be a subset of actual data (i.e., data to be processed using the trained neural network). As a result, there may be the learning cycle during which the error on the training data decreases but the error in the Overfitting is a phenomenon in which the error in the actual data actual data increases due to the excessive learning on the training data. For example, a phenomenon in which a neural network that has trained a cat by looking at a yellow cat does not recognize cats after looking at cats other than yellow may be a kind of overfitting. The overfitting may act as a cause of increasing errors in machine learning algorithms. Various optimization methods may be used to prevent the overfitting. To prevent the overfitting, methods, such as increasing training data, regularization, and dropout that omits some of the nodes in the network during the training process, may be applied

In various embodiments, the computing device 100 may be connected to a user terminal 200 via a network 400, acquire a disease diagnostic request for a specific patient via the user terminal 200, and perform a multiple disease diagnosis based on the biometric data of the patient according to the disease diagnostic request, thereby providing the resulting data to the user terminal 200.

Here, the user terminal 200 is a wireless communication device that ensures portability and mobility, and may include all types of handheld-based wireless communication devices such as a navigation device, a personal communication system (PCS), a global system for mobile communication (GSM), a personal digital cellular (PDC), a personal handyphone system (PHS), a personal digital assistant (PDA), international mobile telecommunications (IMT)-2000, code division multiple access (CDMA)-2000, W-code division multiple access (W-CDMA), a wireless broadband Internet (WiBro) terminal, a smart phone, a smart pad, a tablet personal computer (PC), etc., but are not limited thereto.

In addition, here, the network 400 may be a connection structure capable of exchanging information between respective nodes such as a plurality of terminals and servers. For example, the network 400 may include a local area network (LAN), a wide area network (WAN), the Internet (World Wide Web (WWW)), a wired/wireless data communication network, a telephone network, a wired/wireless television communication network, etc.

In addition, here, examples of the wireless data communication network may include 3G, 4G, 5G, 3^{rd} Generation Partnership Project (3GPP), 5^{th} Generation Partnership Project (5GPP), long term evolution (LTE), world interoperability for microwave access (WiMAX), Wi-Fi, Internet, a local area network (LAN), a wireless local area network (WLAN), a wide area network (WAN), a personal area network (PAN), radio frequency, a Bluetooth network, a near-field communication (NFC) network, a satellite broadcast network, an analog broadcast network, a digital multimedia broadcasting (DMB) network, etc., but are not limited thereto.

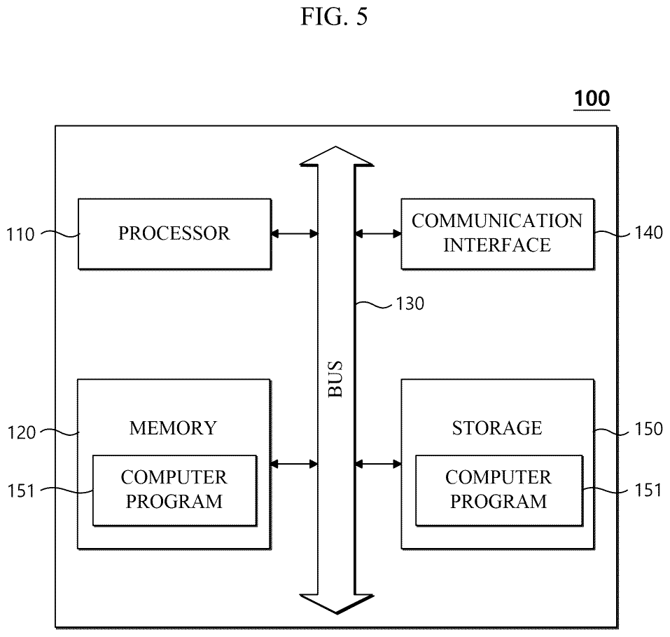

In an embodiment, the external server 300 may be connected to the computing device 100 via the network 400, and may store and manage various types of information and data required for the computing device 100 to perform the digital phenotyping method for drug response classification and prediction, or collect, store, and manage various types of information and data generated as the computing device 100 performs the digital phenotyping method for drug response classification and prediction. For example, the external server 300 may be a storage server separately provided outside the computing device 100 but is not limited thereto. Hereinafter, the hardware configuration of the computing device 100 performing the digital phenotyping method for drug response classification and prediction will be described with reference to FIG. 5.

FIG. 5 is a hardware configuration of a digital phenotyping apparatus for drug response classification and prediction according to another embodiment of the present invention.

Referring to FIG. 5, the computing device 100 may include one or more processors 110, a memory 120 into which a computer program 151 executed by the processor 110 is loaded, a bus 130, a communication interface 140, and a storage 150 for storing the computer program 151. Here, only the components related to the embodiment of the present invention are illustrated in FIG. 5. Accordingly, those skilled in the art to which the present invention pertains may understand that other general-purpose components other than those illustrated in FIG. 5 may be further included.

The processor 110 controls an overall operation of each component of the computing device 100. The processor 110 may include a central processing unit (CPU), a micro processor unit (MPU), a micro controller unit (MCU), a graphic processing unit (GPU), or any type of processor well known in the art of the present invention.

In addition, the processor 110 may perform an operation on at least one application or program for executing the method according to the embodiments of the present invention, and the computing device 100 may include one or more processors.

In various embodiments, the processor 110 may further include a random access memory (RAM) (not illustrated) and a read-only memory (ROM) for temporarily and/or permanently storing signals (or data) processed in the processor 110. In addition, the processor 110 may be implemented in the form of a system-on-chip (SoC) including at least one of a graphics processing unit, a RAM, and a ROM.

The memory 120 stores various data, commands, and/or information. The memory 120 may load the computer program 151 from the storage 150 to execute methods/operations according to various embodiments of the present invention. When the computer program 151 is loaded into the memory 120, the processor 110 may perform the method/operation by executing one or more instructions constituting the computer program 151. The memory 120 may be implemented as a volatile memory such as a RAM, but the technical scope of the present disclosure is not limited thereto.

The bus 130 provides a communication function between the components of the computing device 100. The bus 130 may be implemented as various types of buses, such as an address bus, a data bus, and a control bus.

The communication interface 140 supports wired/wireless Internet communication of the computing device 100. In addition, the communication interface 140 may support various communication manners other than the Internet communication. To this end, the communication interface 140 may include a communication module well known in the art of the present invention. In some embodiments, the communication interface 140 may be omitted.

The storage 150 may non-temporarily store the computer program 151. When performing the digital phenotyping process for drug response classification and prediction through the computing device 100, the storage 150 may store various types of information necessary to provide the digital phenotyping process for drug response classification and prediction.

The storage 150 may include a nonvolatile memory, such as a ROM, an erasable programmable ROM (EPROM), an electrically erasable programmable ROM (EEPROM), and a flash memory, a hard disk, a removable disk, or any well-known computer-readable recording medium in the art to which the present invention pertains.

The computer program 151 may include one or more instructions to cause the processor 110 to perform methods/operations according to various embodiments of the present invention when loaded into the memory 120. That is, the processor 110 may perform the method/operation according to various embodiments of the present invention by executing the one or more instructions.

In an embodiment, the computer program 151 may include one or more instructions to perform a digital phenotyping method for drug response classification and prediction that includes acquiring biometric data of a patient and analyzing the acquired biometric data using a disease diagnostic model to perform a multiple disease diagnosis on the patient.

Operations of the method or algorithm described with reference to the embodiment of the present invention may be directly implemented in hardware, in software modules executed by hardware, or in a combination thereof. The software module may reside in a RAM, a ROM, an erasable programmable ROM (EPROM), an electrically erasable programmable ROM (EEPROM), a flash memory, a hard disk, a removable disk, a compact disc read-only memory (CD-ROM), or in any form of computer-readable recording media known in the art to which the invention pertains.

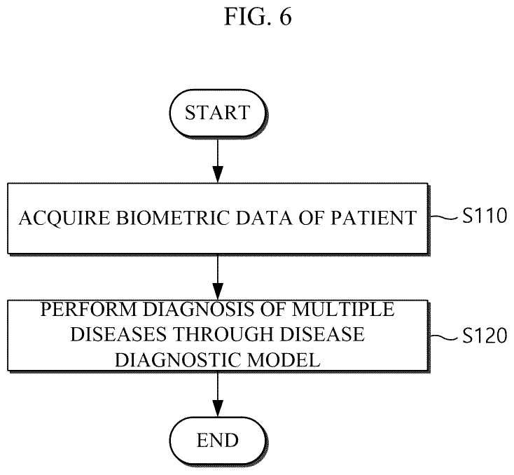

The components of the present invention may be embodied as a program (or application) and stored in media for execution in combination with a computer which is hardware. The components of the present invention may be executed in software programming or software elements, and similarly, embodiments may be realized in a programming or scripting language such as C, C++, Java, and assembler, including various algorithms implemented in a combination of data structures, processes, routines, or other programming constructions. Functional aspects may be implemented in algorithms executed on one or more processors. Hereinafter, the digital phenotyping method for drug response classification and prediction performed by the computing device 100 will be described with reference to FIGS. 6 to 14.

FIG. 6 is a flowchart of a digital phenotyping method for drug response classification and prediction according to a first embodiment of the present invention.

Referring to FIG. 6, in operation S110, the computing device 100 may collect the biometric data of the patient. Here, the biometric data of the patient may be patient's brainwave data but is not limited thereto.

In various embodiments, the computing device 100 may perform a brainwave data collection operation that collects the brainwave data for the patient. For example, the computing device 100 may collect the brainwave data for the patient measured in real time through a brainwave measuring device (not illustrated). However, the computing device 100 is not limited thereto, and may receive brainwave data for a patient previously stored in the external server 300 from the external server 300.

Here, the brainwave data may mean a plurality of pieces of unit brainwave data (e.g., independent brainwave signals measured through each channel) measured through a brainwave measuring device (not illustrated) including a plurality of brainwave measuring channels (e.g., a total of 19 channels (e.g., Fp1, Fp2, F3, F4, C3, C4, P3, P4, O1, O2, F7, F8, T3, T4, T5, T6, Fz, Cz, and Pz)) attached to different locations on a user's head (scalp).

In operation S120, the computing device 100 may analyze the biometric data of the patient (e.g., the patient's brainwave data) acquired through operation S110 to perform a multiple disease diagnosis on the patient.

In various embodiments, the computing device 100 may analyze the biometric data of the patient using the disease diagnostic model to perform a multiple disease diagnosis on the patient. For example, the computing device 100 may generate a disease diagnostic model including a plurality of diagnostic models that independently perform diagnoses for each of multiple distinct diseases based on the biometric data, and derive a probability value which is a possibility for each of multiple distinct diseases for the patient, by inputting the biometric data of the patient to each of the plurality of diagnostic models included in the disease diagnostic model.

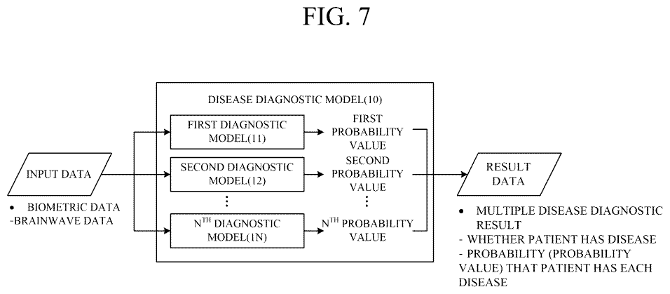

For example, as illustrated in FIG. 7, the computing device 100 may input the biometric data of the patient to each of a plurality of diagnostic models (e.g., a first diagnostic model 11 diagnosing a first disease, a second diagnostic model diagnosing a second disease, and an N^{th} diagnostic model IN diagnosing an N^{th} disease) included in the disease diagnostic model 10, thereby generating a plurality of probability values (e.g., a first probability value that is a possibility of having the first disease, a second probability value that is a possibility of having the second disease, and an N^{th} probability value that is a possibility of having an N^{th} disease), and derive, as result data, multiple disease diagnosis results based on the plurality of generated probability values.

Here, the results of the multiple disease diagnosis for the patient may be the results of determining whether to have each of the multiple diseases based on the plurality of probability values calculated according to the above method, but is not limited thereto, and the results of the multiple disease diagnosis for the patient may be the plurality of probability values calculated by determining the possibility of having each of the multiple diseases.

For example, when the computing device 100 may analyze the biometric data of the patient through the plurality of diagnostic models to calculate the first probability value, which is the possibility of having the first disease, the second probability value, which is the possibility of having the second disease, and the third probability value, which is the possibility of having the third disease, the computing device 100 may compare the first probability value, the second probability value, and the third probability value with the reference probability value to determine whether to have the first disease, the second disease, and the third disease, and provide information on the disease determined to have the disease (the disease whose probability value is greater than or equal to the reference probability value) as the result of the multiple disease diagnosis.

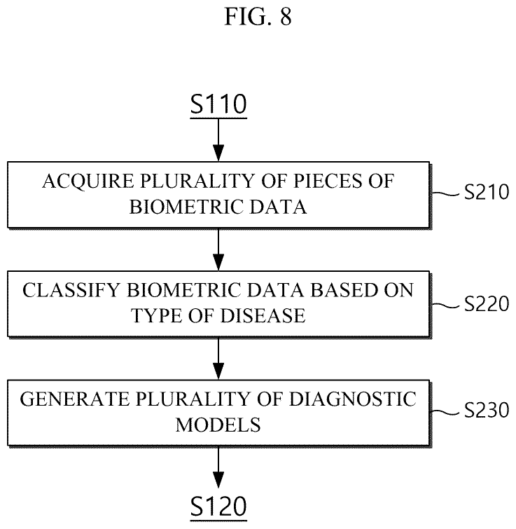

In addition, when the computing device 100 may analyze the biometric data of the patient using the plurality of diagnostic models to calculate the first probability value which is the possibility of having the first disease, the second probability value which is the possibility of having the second disease, and the third probability value which is the possibility of having the third disease, the computing device 100 may provide the first probability value, the second probability value, and the third probability value as the multiple disease diagnosis results. In this case, the computing device 100 may compare the plurality of probability values with the reference probability value to provide only at least one probability value greater than or equal to the reference probability value as the multiple disease diagnosis result. Hereinafter, a method of generating a plurality of diagnostic models performed by the computing device 100 will be described with reference to FIG. 8.

FIG. 8 is a flowchart for describing a method of generating a plurality of diagnostic models in the first embodiment.

Referring to FIG. 8, in operation S210, the computing device 100 may collect a plurality of pieces of biometric data. Here, the plurality of pieces of biometric data may be a plurality of pieces of brainwave data collected from each of multiple patients having different types of brain diseases but is not limited thereto.

In addition, here, the biometric data acquisition operation performed by the computing device 100 may be implemented in a form identical or similar to the operation performed in operation S110 of FIG. 6 but is not limited thereto.

In operation S220, the computing device 100 may classify the plurality of pieces of biometric data acquired through operation S210 according to the type of disease.

In various embodiments, when the computing device 100 acquires a plurality of pieces of brainwave data from multiple patients, the computing device 100 may classify the plurality of pieces of brainwave data according to the type of brain diseases of each of the multiple patients. For example, the computing device 100 may classify the plurality of pieces of brainwave data into brainwave data of pure-ADD, brainwave data of pure-LBD, brainwave data of a Parkinson patient, brainwave data of a vascular dementia patient, brainwave data of a depression patient, etc., but is not limited thereto.

In various embodiments, the computing device 100 may primarily classify the plurality of pieces of brainwave data according to attributes (e.g., basic profiles such as an age and gender of a patient) of a patient so that normality is high and secondarily classify the brainwave data classified primarily according to the type of diseases.

In operation S230, the computing device 100 may generate a plurality of diagnostic models that individually diagnose whether to have different diseases by using the plurality of biometric data classified through operation S220 as the training data to train different diagnostic models. For example, the computing device 100 may train different diagnostic models using each of pieces of brainwave data classified as ADD, LBD, Parkinson, vascular dementia, and depression as training data to generate a diagnostic model for diagnosing the ADD, a diagnostic model for diagnosing the LBD, a diagnostic model for diagnosing the Parkinson, a diagnostic model for diagnosing the vascular dementia, and a diagnostic model for diagnosing the depression.

In various embodiments, the computing device 100 may label information (e.g., disease names) about diseases of multiple patients for each of the plurality of pieces of brainwave data to generate the training data and may train a diagnostic model according to a supervised learning method using the training data, but is not limited thereto.

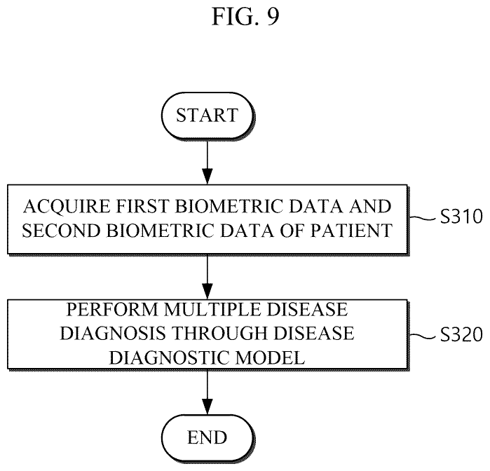

FIG. 9 is a flowchart of a digital phenotyping method for drug response classification and prediction according to a second embodiment of the present invention.

Referring to FIG. 9, in operation S310, the computing device 100 may acquire first biometric data of a patient at a first time point and second biometric data of a patient at a second time point.

Here, the first time point may mean a time point before the patient takes a target drug, and the second time point may mean a time point after the patient takes the target drug. That is, the first biometric data of the patient at the first time point may mean the biometric data of the patient at the first time point when the target drug is not taken, and the second biometric data of the patient at the second time point may mean biometric data collected at a certain time point after the patient takes the target drug after the first time point, but is not limited thereto.

For example, the computing device 100 may acquire first biometric data of a patient measured at time point t1 and then acquire second biometric data of a patient measured at time point t2 after the patient takes the target drug. Here, the target drug may be a drug for treating a specific disease. For example, the target drug may be, but is not limited to, a drug for ADD, a drug for LBD, or a drug for depression.

In various embodiments, the computing device 100 may acquire the plurality of pieces of first biometric data and the plurality of pieces of second biometric data.

Here, the plurality of pieces of first biometric data and the plurality of pieces of second biometric data may be, but are not limited to, the plurality of pieces of brainwave data collected from each of the plurality of patients having different types of brain diseases.

In addition, here, the biometric data acquisition operation performed by the computing device 100 here may be implemented in a form identical or similar to the operation performed in operation S110 of FIG. 6 but is not limited thereto.

In operation S320, the computing device 100 may analyze the pieces of first biometric data acquired through operation S310 using the disease diagnostic model to perform the multiple disease diagnosis on the patient. Here, the multiple disease diagnosis operation performed by the computing device 100 may be implemented in a form identical or similar to the operation performed in operation S120 of FIG. 6 but is not limited thereto.

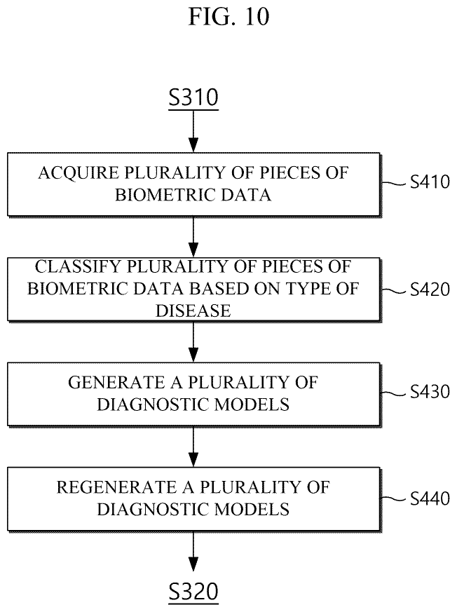

FIG. 10 is a flowchart for describing a method of generating a plurality of diagnostic models in the second embodiment.

Referring to FIG. 10, in operation S410, the computing device 100 may acquire the first brainwave data measured at the first time point and the second brainwave data measured at the second time point for each of the multiple patients as the biometric data of multiple patients having different types of brain diseases.

In addition, here, the biometric data acquisition operation performed by the computing device 100 may be implemented in a form identical or similar to the operation performed in operation S110 of FIG. 6 but is not limited thereto.

In operation S420, the computing device 100 may classify the first brainwave data acquired through operation S410.

In various embodiments, when the computing device 100 acquires a plurality of pieces of first brainwave data from a plurality of patients, the computing device 100 may classify the plurality of pieces of first brainwave data according to the type of brain disease of each of the plurality of patients. For example, the computing device 100 may classify the plurality of pieces of brainwave data into brainwave data of pure-ADD, brainwave data of pure-LBD, brainwave data of a Parkinson patient, brainwave data of a vascular dementia patient, brainwave data of a depression patient, etc., but is not limited thereto.

In operation S430, the computing device 100 may generate a plurality of diagnostic models that individually diagnose whether to have different types of brain diseases by using the first brainwave data classified through operation S420 as the training data to train different diagnostic models. For example, the computing device 100 may train different diagnostic models using each of pieces of brainwave data classified as ADD, LBD, Parkinson, vascular dementia, and depression as training data to generate a diagnostic model for diagnosing the ADD, a diagnostic model for diagnosing the LBD, a diagnostic model for diagnosing the Parkinson, a diagnostic model for diagnosing the vascular dementia, and a diagnostic model for diagnosing the depression.

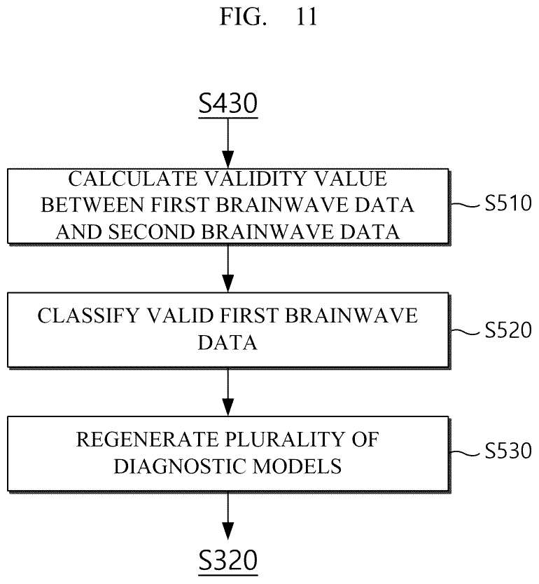

In operation S440, the computing device 100 may regenerate a plurality of diagnostic models generated through operation S430. Hereinafter, a method for regenerating a plurality of diagnostic models will be specifically described with reference to FIG. 11.

FIG. 11 is a flowchart explaining a sequence for regenerating a plurality of diagnostic models in the second embodiment.

Referring to FIG. 11, in operation S510, the computing device 100 may calculate a validity value through the comparison between the acquired first brainwave data and the acquired second brainwave data. Here, the second brainwave data may be information reflecting a prognosis of the target drug of the patient after acquiring the first brainwave data of the patient.

For example, the computing device 100 may compare an Alpha peak frequency value of the first brainwave data with an Alpha peak frequency value of the second brainwave data to calculate the difference value as the validity value.

As another example, the computing device 100 may statistically compare a first brainwave data value with a second brainwave data value to calculate a p-value. Here, the comparison of the first brainwave data and the second brainwave data is not limited to the above example and may include comparing all data acquired from the brainwave data and metadata generated by analyzing the corresponding brainwave data.

In operation S520, the computing device 100 may classify the acquired first brainwave data as valid first brainwave data when the validity value is greater than or equal to the preset validity value. For example, when the validity value calculated through S510 is greater than or equal to a cut off value, the computing device 100 may classify the first brainwave data acquired through operation S510 as the valid first brainwave data. For example, when the 1/p-value value acquired by statistically comparing the first brainwave data value and the second brainwave data value is greater than or equal to 33.3, the computing device 100 may classify the first brainwave data of the corresponding patient as the valid first brainwave data. However, this is only one example for classifying the valid first brainwave data and is not limited to this example.

In operation S530, the computing device 100 may regenerate the plurality of diagnostic models using the valid first brainwave data as the training data. For example, the computing device 100 may regenerate the plurality of diagnostic models based on the valid first brainwave data so as to replace a plurality of diagnostic models previously generated based on the first brainwave data.

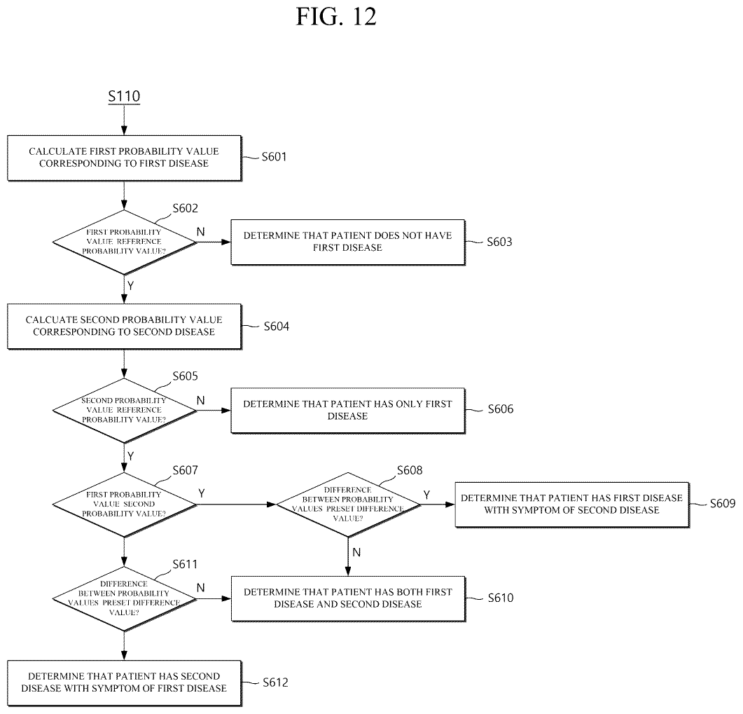

In addition, the computing device 100 may train a diagnostic model using biometric data of a patient with disease and biometric data of a normal person without disease as training data, thereby generating a plurality of diagnostic models that classify patients with disease and normal people. Hereinafter, various multiple disease diagnosis methods performed through a plurality of diagnostic models will be specifically described with reference to FIGS. 12 to 14

FIG. 12 is a flowchart for describing a method of sequentially performing a diagnosis of a first disease and a second disease having a mutual correlation in various embodiments.

Referring to FIG. 12, in operation S601, when the computing device 100 acquires a first disease diagnostic request (e.g., an ADD diagnosis request) for a patient from a user, the computing device 100 analyzes the biometric data of the patient through the first diagnostic model that diagnoses the first disease, thereby calculating the first probability value corresponding to the first disease, i.e., the first probability value that the patient has the first disease.

Here, the user may be a medical professional who wants to diagnose the disease of the patient, but is not limited thereto, and the user may be a patient's guardian or a patient himself.

In operation S602, the computing device 100 may compare the first probability value calculated through operation S601 with the reference probability value to determine whether the first probability value is greater than or equal to the reference probability value.

Here, the reference probability value is a probability value that serves as a reference for determining whether the patient has the first disease, and may be a preset value (e.g., 0.5), but is not limited thereto.

In operation S603, when the computing device 100 determines that the first probability value is less than the reference probability value as a result of comparing the first probability value with the reference probability value through operation S602, the computing device 100 may determine that the patient does not have the first disease, i.e., the patient is a normal person who does not have the first disease.

In operation S604, when the computing device 100 determines that the first probability value is greater than or equal to the reference probability value as a result of comparing the first probability value with the reference probability value through operation S602, the computing device determines that the patient has the first disease and at the same time, analyzes the biometric data of the patient using the second diagnostic model that diagnoses the second disease, thereby calculating the second probability value corresponding to the second disease, that is, the second probability value that is the possibility that the patient has the second disease.

Here, the second disease may be a disease that is correlated with the first disease. For example, when the first disease is ADD, the second disease may be LBD that is correlated with ADD but is not limited thereto.

In operation S605, the computing device 100 may compare the second probability value calculated through operation S604 with the reference probability value to determine whether the second probability value is greater than or equal to the reference probability value.

Here, the reference probability value is a preset value as a reference for determining whether the patient has the second disease, and may be the same value (e.g., 0.5) as the reference probability value for determining whether the patient has the first disease but is not limited thereto.

In operation S606, when the computing device 100 determines that the second probability value is less than the reference probability value as a result of comparing the second probability value with the reference probability value through operation S605, that is, when the computing device 100 determines that the first probability value is greater than or equal to the reference probability value and the second probability value is less than the reference probability value, the computing device may determine that the patient has only the first disease. For example, when the first disease is ADD, the computing device 100 may determine that the patient is a patient with only ADD (e.g., a pure-ADD).

Meanwhile, when the computing device 100 determines that the second probability value is greater than or equal to the reference probability value as a result of comparing the second probability value with the reference probability value through operation S605, that is, when the computing device 100 determines that both the first probability value and the second probability value are greater than or equal to the reference probability value, the computing device may determine that the patient has both the first disease and the second disease. For example, when the first disease is ADD and the second disease is LBD, it may be determined that the patient has ADD LBD mixed.

In this case, as described above, the LBD has the characteristics of faster disease progression and greater decline in cognitive function compared to the ADD, and the characteristics differ depending on which type of dementia has a higher proportion between the LBD and the ADD. Therefore, it is possible to determine the dominant of the ADD and the LBD based on a comparison result of magnitudes of the first and second probability values and the difference therebetween in addition to simply determining that the patient has both the LBD and the ADD, thereby diagnosing the patient's conditions more specifically (operations S607 to S612 to be described below).

In operation S607, when the computing device 100 determines that the second probability value is greater than or equal to the reference probability value as a result of comparing the magnitudes of the second probability value and the reference probability value through operation S605, the computing device 100 may perform a comparison of the magnitudes of the first probability value and the second probability value for the purpose of determining the dominant of the first disease and the second disease.

In operation S608, when the computing device 100 determines that the first probability value is greater than the second probability value as a result of comparing the magnitudes of the first probability value and the second probability value through operation S607, the computing device 100 may determine whether the difference between the first probability value and the second probability value is greater than or equal to a preset difference value.

Here, the preset difference value may be a reference for distinguishing the dominant between the first disease and the second disease, and may be a preset value (e.g., 0.2) but is not limited thereto.

In operation S609, when the computing device 100 determines through operation S608 that the difference between the first probability value and the second probability value is greater than or equal to the preset difference value, that is, when the computing device determines that the first probability value is greater than the second probability value by the preset difference value or greater, the computing device 100 may determine that the first disease may be dominant over the second disease, and thus, determine that the patient is a patient with the first disease mixed with symptoms of the second disease (e.g., a patient with ADD mixed with symptoms of the LBD (ADD dominant LBD mix)).

In operation S610, when the computing device 100 determines through operation S608 that the difference between the first probability value and the second probability value is less than the preset difference value, the computing device 100 may determine that there is no dominant disease between the first disease and the second disease, and thus, determine that the patient is a patient with both the first disease and the second disease (e.g., a patient with both the LBD and the ADD (ADD LBD mix)).

In operation S611, when the computing device 100 determines that the second probability value is greater than the first probability value as a result of comparing the magnitudes of the first probability value and the second probability value through operation S607, the computing device 100 may determine whether the difference between the second probability value and the first probability value is greater than or equal to the preset difference value.

In this case, when the computing device 100 determines that the difference between the second probability value and the first probability value is less than the preset difference value, as in operation S610, the computing device 100 may determine that there is no dominant disease between the first disease and the second disease, and thus, determine that the patient is a patient with both the first disease and the second disease (e.g., a patient with both the LBD and the ADD (ADD LBD mix)).

In operation S612, when the computing device 100 determines through operation S611 that the difference between the first probability value and the second probability value is greater than or equal to the preset difference value, the computing device 100 may determine that the second disease is a dominant disease compared to the first disease, and thus, determine that the patient is a patient with the second disease mixed with symptoms of the first disease (e.g., a patient with the LBD mixed with symptoms of the ADD (LBD dominant ADD mix)).

That is, as described above, the computing device 100 diagnoses whether the patient has two or more diseases by using two or more probability values derived through two or more diagnostic models that diagnose two or more diseases that are correlated, and determines the dominant for the two or more diseases based on the comparison of the magnitudes of the two or more probability values corresponding to the two or more diseases and the difference value therebetween to diagnose the patient's conditions, so it may more specifically diagnose not only whether the patient has two or more diseases, but also which disease the patient dominantly has.

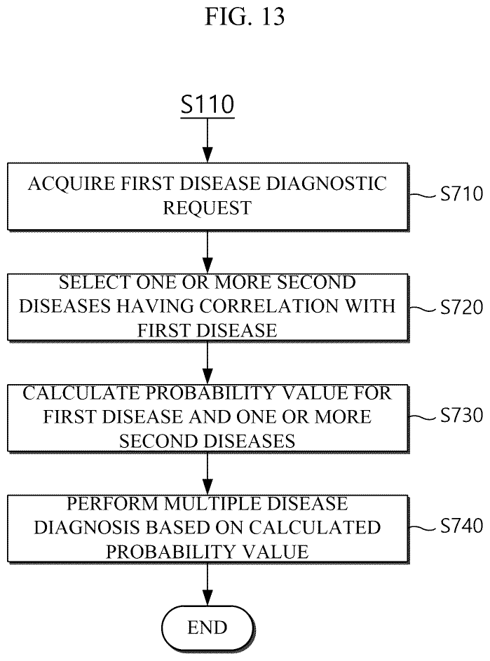

FIG. 13 is a flowchart for describing a method of simultaneously performing a diagnosis of a first disease and a second disease having a correlation in various embodiments.

Referring to FIG. 13, in operation S710, the computing device 100 may acquire a first disease diagnostic request for a patient from a user. For example, the computing device 100 may be connected to the user terminal 200 through the network 400, and may acquire the first disease diagnostic request while acquiring the biometric data of the patient through the user terminal 200, but is not limited thereto.

In operation S720, the computing device 100 may select one or more second diseases that are correlated with the first disease. For example, when the computing device 100 acquires a request for ADD diagnosis for the patient from the user, the computing device may select the LBD that is correlated with the ADD. Conversely, when acquiring a request for LBD diagnosis for a patient from a user, the ADD, which is correlated with the LBD, may be selected.

In various embodiments, the computing device 100 may predefine the correlation between multiple diseases, and when acquiring a request for diagnosis of a specific disease from a user, select at least one disease having the correlation with the specific disease based on a plurality of predefined correlations between diseases.

In operation S730, the computing device 100 may calculate the first probability value which is the possibility of having the first disease, and one or more second probability values which are the possibility of having one or more second diseases.

In various embodiments, the computing device 100 may analyze the biometric data of the patient through one diagnostic model diagnosing the first disease and one or more diagnostic models diagnosing one or more second diseases among the plurality of diagnostic models to calculate the first probability value, which is the possibility of having the first disease, and one or more second probability values which are the possibility of having one or more second diseases.

In operation S740, the computing device 100 may perform the multiple disease diagnosis on the patient based on the probability values (the first probability value and one or more second probability values) calculated through operation S430.

For example, the computing device 100 may determine whether the first probability value and one or more second probability values are greater than or equal to the reference probability value to determine whether the patient has the first disease and one or more second diseases and derive the results of the multiple disease diagnosis including the determination result.

In addition, when the computing device 100 determines that the first probability value and one or more second probability values are greater than or equal to the reference probability value, the computing device 100 may determine the dominant between the first disease and one or more second diseases based on the result of comparing the magnitudes of the first probability value and one or more second probability values and the difference between the first probability value and one or more second probability values, diagnose the patient's condition according to the determination result, and derive the results of the multiple disease diagnosis including the diagnosis result.

As another example, the computing device 100 may derive the first probability value and one or more second probability values as the results of the multiple disease diagnosis. In this case, only the probability values, which are greater than or equal to a reference probability value, among the first probability value and one or more second probability values may be derived as the results of the multiple disease diagnosis.

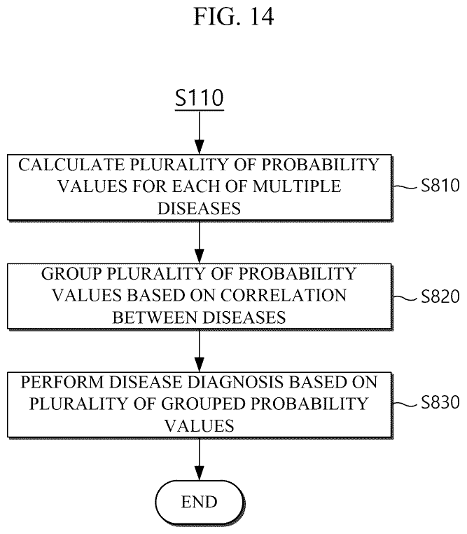

FIG. 14 is a flowchart for describing a method of simultaneously performing a diagnosis of multiple diseases in various embodiments

Referring to FIG. 14, in operation S810, the computing device 100 may analyze the biometric data of the patient to calculate the plurality of probability values that are the possibilities that the patient may have each of the multiple diseases.

In various embodiments, the computing device 100 may analyze the biometric data of the patient through each of the plurality of diagnostic models that individually diagnose each of the multiple different types of diseases, thereby calculating the plurality of probability values that are possibilities that the patient may have each of the multiple diseases.

In operation S820, the computing device 100 may predefine the correlation between the multiple diseases and may classify and group the plurality of probability values according to the correlations between diseases based on the plurality of predefined correlations between diseases, thereby generating a plurality of groups.

Here, the probability values included in each of the plurality of groups may include probability values which are the possibilities that the patient may have diseases that have the correlations. For example, the computing device 100 may group the first probability value which is the possibility of having the ADD, and the second probability value which is the possibility of having the LBD correlated with the ADD, among the plurality of probability values, to generate one group, but is not limited thereto.

In operation S830, the computing device 100 may perform the disease diagnosis based on the plurality of grouped probability values.

For example, when the first probability value, which is the possibility of having the ADD, and the second probability value, which is the possibility of having the LBD, are included in one group, the computing device 100 may determine whether the first probability value and the second probability value are greater than or equal to the reference probability value to determine whether the patient has the ADD and the LBD and derive the results of the multiple disease diagnosis including the determination result.

In addition, when the computing device 100 determines that the first probability value and one or more second probability values are greater than or equal to the reference probability value, the computing device 100 may determine the dominant between the AMD and the LBD based on the comparison result of the magnitudes of the first probability value and one or more second probability values and the difference between the first probability value and one or more second probability value, diagnose (e.g., a patient with ADD mixed with symptoms of the LBD, a patient with LBD mixed with symptoms of the ADD, ADD LBD mixed, etc.) the patient's condition according to the determination result, and derive the results of the multiple disease diagnosis including the diagnosis result.

As another example, the computing device 100 may derive the first probability value, which is the possibility of having the ADD, and the second probability value, which is the possibility of having the LBD, as the results of the multiple disease diagnosis. In this case, only the probability value, which is greater than or equal to the reference probability value, among the first probability value and the second probability value may be derived as the results of the multiple disease diagnosis.

The above-described digital phenotyping method for drug response classification and prediction has been described with reference to the flowchart illustrated in the drawings. For a simple description, the digital phenotyping method for drug response classification and prediction has been described by illustrating a series of blocks, but the present invention is not limited to the order of the blocks, and some blocks may be performed in an order different from that shown and performed in the present specification or may be performed concurrently. In addition, new blocks not described in the present specification and drawings may be added, or some blocks may be omitted or changed.

According to various embodiments of the present invention, by analyzing the biometric data of the patient to determine whether the patient has ADD, and by determining not only whether the patient has ADD but also whether the patient has dementia mixed with LBD, it is possible to accurately classify the type of dementia of the dementia patient.

In addition, by analyzing the biometric data of the patient through the plurality of different diagnostic models, it is possible to independently and simultaneously perform not only whether the patient has dementia, but also different types of brain diseases such as LBD, Parkinson, vascular dementia, depression, and anxiety.

Effects of the present invention are not limited to the effects described above, and other effects that are not mentioned may be obviously understood by those skilled in the art from the following description.

Although exemplary embodiments of the present invention have been described with reference to the accompanying drawings, those skilled in the art will understand that various modifications and alterations may be made without departing from the spirit or essential feature of the present invention. Therefore, it is to be understood that the exemplary embodiments described hereinabove are illustrative rather than being restrictive in all aspects.

What is claimed is:

1. A digital phenotyping method for drug response classification and prediction, which is performed by a computing device, the method comprising:
acquiring biometric data of a patient; and
performing a multiple disease diagnosis on the patient by analyzing the acquired biometric data using a disease diagnostic model,
wherein the disease diagnostic model includes a plurality of diagnostic models that independently perform diagnoses of each of multiple distinct diseases based on the acquired biometric data.

2. The digital phenotyping method of claim 1, further comprising:
acquiring a plurality of pieces of brainwave data for each of multiple patients as biometric data of the multiple patients having different types of brain diseases;
classifying the plurality of pieces of acquired brainwave data based on a type of brain disease; and
generating a plurality of diagnostic models that individually diagnose whether to have different types of brain diseases by training different diagnostic models using the plurality of pieces of classified brainwave data as training data.

3. The digital phenotyping method of claim 2, wherein the acquiring of the plurality of pieces of brainwave data includes:
acquiring first brainwave data for a patient with a specific brain disease at a first time point, which is a time point before the patient with the specific brain disease takes a target drug; and
acquiring second brainwave data for the patient with the specific brain disease at a second time point, which is a time point after the patient with the specific brain disease takes the target drug, and
the generating of the plurality of diagnostic models includes:
comparing the acquired first brainwave data with the acquired second brainwave data to calculate a validity value;
classifying the acquired first brainwave data into valid first brainwave data when the calculated validity value is greater than or equal to a preset validity value; and
training a diagnostic model, which diagnoses whether the patient has the specific brain disease, among the plurality of generated diagnostic models using the classified valid first brainwave data as the training data.

4. The digital phenotyping method of claim 1, wherein the plurality of diagnostic models include a first diagnostic model for diagnosing whether a first disease is present and a second diagnostic model for diagnosing whether a second disease related to the first disease is present, and
the performing of the multiple disease diagnosis includes:
calculating a first probability value, which is a possibility that the patient has the first disease, by analyzing the acquired biometric data through the first diagnostic model, when a request for diagnosis of the first disease for the patient is acquired from a user, and
when the calculated first probability value is greater than or equal to a reference probability value, calculating a second probability value, which is a possibility that the patient has the second disease, by analyzing the acquired biometric data through the second diagnostic model; and
performing multiple diagnoses of whether the first disease is present and whether the second disease is present based on the calculated first probability value and the calculated second probability value.

5. The digital phenotyping method of claim 4, wherein the performing of the multiple diagnoses includes:
determining that the patient has only the first disease when the calculated second probability value is less than the reference probability value; and
determining that the patient has the first disease and the second disease when the calculated second probability value is greater than or equal to the reference probability value.

6. The digital phenotyping method of claim 5, wherein the determining of that the patient has the first disease and the second disease includes:
determining a dominant between the first disease and the second disease based on a result of comparing magnitudes of the calculated first probability value and the calculated second probability value and a difference between the calculated first probability value and the calculated second probability value;
determining that the patient has the first disease mixed with symptoms of the second disease based on the determined dominant, when the calculated first probability value is greater than the calculated second probability value and the difference between the calculated first probability value and the calculated second probability value is greater than or equal to a preset difference value;
determining that the patient has both the first disease and the second disease based on the determined dominant when a magnitude of the difference between the calculated first probability value and the calculated second probability value is less than the preset difference value; and
determining that the patient has the second disease mixed with symptoms of the first disease based on the determined dominant, when the calculated second probability value is greater than the calculated first probability value and a difference between the calculated second probability value and the calculated first probability value is greater than or equal to the preset difference value.

7. The digital phenotyping method of claim 1, wherein the performing of the multiple disease diagnosis includes:
calculating a probability value corresponding to the possibility that the patient has each of the multiple distinct diseases by inputting the acquired biometric data to each of the plurality of diagnostic models; and
selecting at least one disease of which a calculated probability value is greater than or equal to a reference probability value from among the multiple distinct diseases, and determining that the patient is a patient having at least one of the selected diseases as a result of the multiple disease diagnosis of the patient.

8. The digital phenotyping method of claim 1, wherein the performing of the multiple disease diagnosis includes:
selecting at least one second disease having a correlation with the first disease based on a plurality of predefined correlations between diseases when acquiring a first disease diagnostic request for the patient from a user; and
calculating a first probability value that is a possibility of having the first disease and one or more second probability values that is a possibility of having the selected one or more second diseases by analyzing the acquired biometric data through one diagnostic model that performs a diagnosis of the first disease among the plurality of diagnostic models and one or more diagnostic models that perform a diagnosis of the selected one or more second diseases.

9. The method of claim 1, wherein the performing of the multiple disease diagnosis includes:
calculating a plurality of probability values, which are possibilities of having each of the multiple distinct diseases, by analyzing the acquired biometric data through the plurality of diagnostic models;
grouping the plurality of calculated probability values according to correlations based on a plurality of predefined correlations between diseases; and
performing the multiple disease diagnosis on the patient based on a comparison result of magnitudes of each of the plurality of grouped probability values and a reference probability value, a comparison result of a magnitude between the plurality of grouped probability values, and a difference between the plurality of grouped probability values.

10. A digital phenotyping method for drug response classification and prediction, which is performed by a computing device, the method comprising:
acquiring first biometric data of a patient at a first time point which is a time point before the patient takes a target drug;
acquiring second biometric data of the patient at a second time point which is later than the first time point and is a time point after the patient takes the target drug; and
analyzing the acquired first biometric data using a disease diagnostic model to perform a multiple disease diagnosis on the patient,
wherein the disease diagnostic model includes a plurality of diagnostic models that independently perform diagnoses of each of multiple distinct diseases based on the acquired biometric data.

11. The digital phenotyping method of claim 10, further comprising:
acquiring first brainwave data measured at the first time point and second brainwave data measured at the second time point for each of the multiple patients as biometric data of multiple patients having different types of brain diseases;
classifying the acquired first brainwave data based on a type of brain disease; and
generating a plurality of diagnostic models that individually diagnose whether different types of brain diseases are present by training different diagnostic models using the classified first brainwave data as training data.

12. The digital phenotyping method of claim 11, further comprising:
calculating a validity value through a comparison between the acquired first brainwave data and second brainwave data;
classifying the acquired first brainwave data into valid first brainwave data when the calculated validity value is greater than or equal to a preset validity value; and
regenerating the plurality of diagnostic models using the valid first brainwave data as the training data.

13. A digital phenotyping apparatus for drug response classification and prediction, comprising:
a processor;
a network interface;
a memory; and
a computer program loaded into the memory and executed by the processor,
wherein the computer program includes:
an instruction for acquiring biometric data of a patient; and
an instruction for analyzing the acquired biometric data using a disease diagnostic model to perform a multiple disease diagnosis on the patient, and
the disease diagnostic model includes a plurality of diagnostic models that independently perform diagnoses of each of multiple distinct diseases based on the acquired biometric data.

14. A computer-readable recording medium, on which a computer program which is combined with a computing device to execute a digital phenotyping method for drug response classification and prediction, wherein the digital phenotyping method includes:
acquiring biometric data of a patient; and
analyzing the acquired biometric data using a disease diagnostic model to perform a multiple disease diagnosis on the patient, the disease diagnostic model including a plurality of diagnostic models that independently perform diagnoses of each of multiple distinct diseases based on the acquired biometric data.
